(19) United States
(12) Patent Application Publication
(10) Pub. No.: US 2012/0059301 A1
(43) Pub. Date: Mar. 8, 2012

(54) Therapuetic Diffusion Hydrocolloid Wound Dressings with Methods of Oxygen Level Indication

(76) Inventor: Amie B. Franklin, Las Vegas, NV (US)

(21) Appl. No.: 13/221,122

(22) Filed: Aug. 30, 2011

Related U.S. Application Data
(60) Provisional application No. 61/378,045, filed on Aug. 30, 2010.

(51) Int. Cl. A61F 13/02

(52) U.S. Cl. 602/48

(57) ABSTRACT

A wound dressing has a reservoir between a not-permeable upper layer, and a lower permeable layer, an adhesion layer facing in the direction of the permeable layer, to be pressed to skin of a patient around a wound, sealing the dressing to the patient, and one or the other of an oxygen-level indicator containing a material exhibiting color change with varying concentration of oxygen, the indicator placed to be visible to a person viewing the dressing through an interface not permeable to oxygen, and to be open to oxygen through a second interface in intimate contact with oxygen in the reservoir, or a self-sealing opening through which a hollow tube may pass without allowing oxygen to escape, the self-sealing opening useful for charging the reservoir with oxygen at pressure, or for sampling oxygen in the reservoir by an external probe.

### BACKGROUND OF THE INVENTION

1. Field of the Invention

The present invention relates to wound dressings, but more particularly to wound dressings that can diffuse oxygen and indicate level of oxygen present.

2. Description of Related Art

The role of oxygen in wound healing has been intensively studied, and is well-known in the art. The present inventor has filed several patent applications in the US and foreign jurisdictions relating to tissue dressings that are capable of providing oxygen to wounds at relatively high concentrations without a need for producing oxygen chemically, electrochemically or from a tethered source. In addition, such tissue dressings are known to the inventor (not necessarily public domain) that allow for delivery of other gases at predetermined gas ratios to tissues and other target areas. This simple means to manipulate and optimize local environments can be used alone or in combination with other materials and/or devices to create additive and sometimes synergistic outcomes (e.g., heat, electrical stimulation, growth factors, and nutrients), or using oxygen in combination with antibiotics topically to enhance antimicrobial effectiveness.

In the area of oxygen supply by a tissue dressing, particularly wherein oxygen is supplied from a reservoir of the dressing through, for example, a permeable membrane, there is an unmet need. It is not intuitive for the person to whom the dressing is applied, or to medical personnel who might be responsible for monitoring use of such dressings with patients under their charge, to know whether or not an original supply of oxygen has been used up, requiring that a dressings be replaces, or in some instances perhaps recharges.

Therefore there is a clear need in the art for apparatus and methods to monitor the useful oxygen level of tissue dressings and the like that are intended to deliver oxygen to a wound, or for other beneficial purpose.

### SUMMARY

In one embodiment a therapeutic wound dressing is provided, comprising a reservoir having a two-dimensional footprint formed between an upper layer not permeable to gases, and a lower layer permeable to gases, an adhesion layer overlapping periphery of the upper, non-permeable layer and extending beyond periphery of the two-dimensional footprint, the adhesion material facing in the direction of the permeable layer, such that the adhesion layer may be pressed to skin of a patient around a wound, sealing the dressing to the patient with the permeable layer directly over the wound, and one or the other of an oxygen-level indicator containing a material exhibiting color change with varying concentration of oxygen, the indicator placed to be visible to a person viewing the dressing through an interface not permeable to oxygen, and to be open to oxygen through a second interface in intimate contact with oxygen in the reservoir, or a self-sealing opening through which a hollow tube may pass without allowing oxygen to escape, the self-sealing opening useful for charging the reservoir with oxygen at pressure, or for sampling oxygen in the reservoir by an external probe.

In one embodiment of the wound dressing the oxygen level indicator comprises a gelatinous material Methylene Blue. Also in one embodiment the oxygen level indicator is a strip of material also comprising a color chart indication oxygen concentration relative to color. In another embodiment the wound dressing is packaged between two layers of release paper for storage or transport.

In another aspect of the invention a method for monitoring oxygen level in an oxygen reservoir of a therapeutic wound dressing is provided, comprising the steps of (a) positioning one or the other of an oxygen level indicator containing a material exhibiting color change with varying concentration of oxygen, the indicator placed to be visible to a person viewing the dressing through an interface not permeable to oxygen, and to be open to oxygen through a second interface in intimate contact with oxygen in the reservoir, or a self-sealing opening into the reservoir through which a hollow tube may pass without allowing oxygen to escape; (b) if the dressing comprises the visible oxygen-level indicator, viewing the indicator and comparing color of the indicator with a reference to determine oxygen level in the reservoir; and (c) if the reservoir comprises the self-sealing opening, passing a hollow probe tip through the opening, collecting a sample of atmosphere in the reservoir for analysis for oxygen concentration.

In one embodiment of the method the oxygen level indicator comprises a gelatinous material Methylene Blue. Also in one embodiment the oxygen level indicator is a strip of material also comprising a color chart indication oxygen concentration relative to color. In some cases the wound dressing is packaged between two layers of release paper for storage or transport.

### BRIEF DESCRIPTION OF THE SEVERAL VIEWS OF THE DRAWINGS

FIG. 1 is an isometric view of a therapeutic diffusion wound dressing according to one embodiment of the present invention.

FIG. 2 is an isometric view of a therapeutic diffusion wound dressing according to another embodiment of the present invention.

FIG. 3 is a partial cross-sectioned view of the structure of the therapeutic diffusion wound dressing of FIG. 3 according to one embodiment of the present invention.

FIG. 4 is an isometric view of a therapeutic diffusion wound dressing and a device to analyze the oxygen content of the wound dressing according to one embodiment of the present invention.

### DETAILED DESCRIPTION OF THE INVENTION

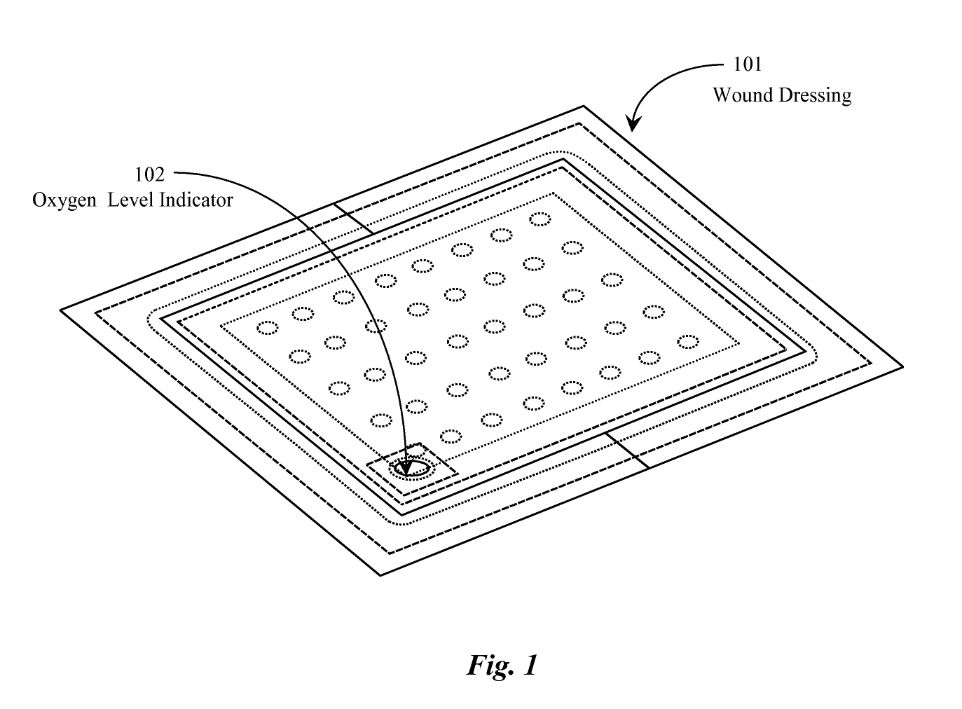

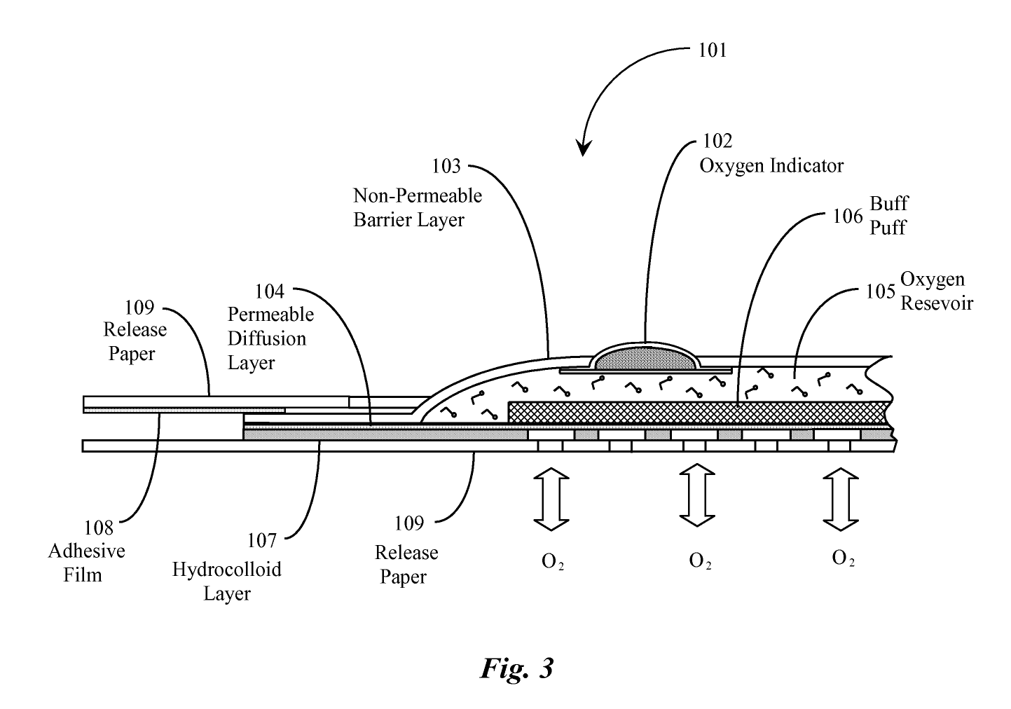

FIG. 1 illustrates a therapeutic diffusion hydrocolloid wound dressing 101 that is designed to deliver elevated levels of oxygen to a wound site for purposes of accelerated healing. Dressing 101 in one embodiment comprises an oxygen reservoir created by sandwiching a layer of open material (buff puff) between a permeable diffusion layer and a non-permeable barrier layer. The permeable diffusion layer may be placed over the wound site and held in place at its perimeter with an adhesive film border and a hydrocolloid layer that makes direct contact to the skin of the wound site. The hydrocolloid layer provides an additional seal that prevents loss of therapeutic gas from the reservoir of the dressing. The adhesive film and hydrocolloid layer should be strong enough to keep the film of the dressing in place for a period of up to five days without allowing air channels to form, whereby therapeutic oxygen may escape. The dressing may also have an upper and/or a lower release paper component that provides protection to the adhesive films and hydrocolloid layer, while also providing an easier method for application to a wound site. The layered structure that makes up the therapeutic diffusion wound dressing, according to one embodiment of the present invention, is illustrated in FIG. 3 of this disclosure.

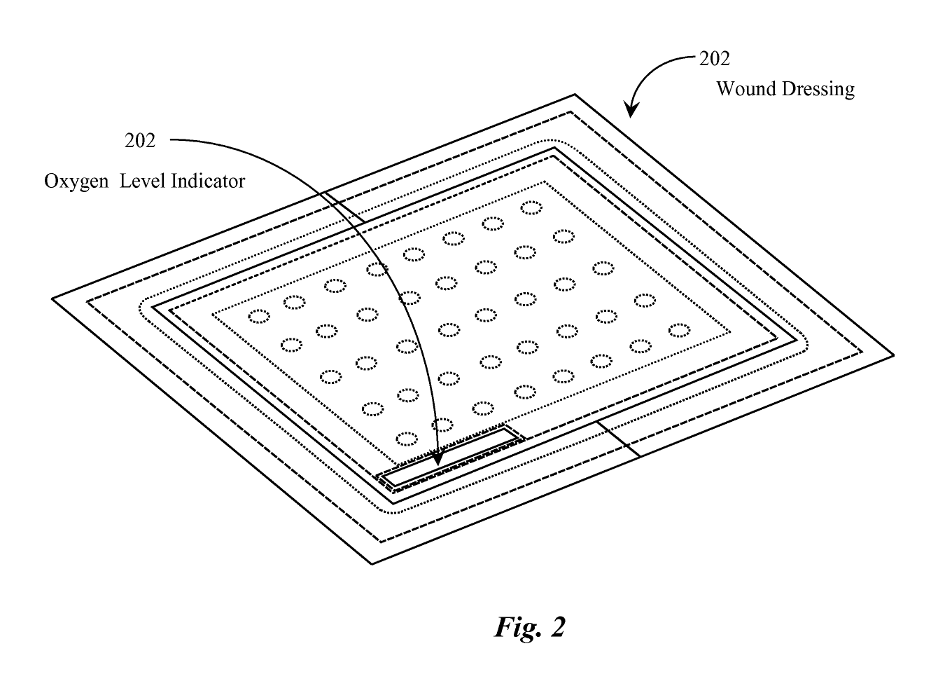

The therapeutic diffusion wound dressings, illustrated in FIG. 1 and FIG. 2 of this disclosure, also comprise an oxygen level indicator (102 in FIGS. 1 and 202 in FIG. 2) incorporated into the non-permeable barrier layer of the oxygen reservoir. It is well known to the medical industry that oxygen levels that fall below about twenty-one percent can provide a growth environment for anaerobic bacteria, or other conditions such as hypoxia. These common conditions, especially beneath a wound dressing, often retard the healing process of the wound site. The inventor of the present disclosure has determined that by incorporating an easily identifiable marker to the bandage, medical personnel or patients may readily determine when the oxygen level of the diffusion wound dressing falls to an unacceptable level. The dressing may then be replaced with a fresh one, or in other embodiments of the therapeutic diffusion wound dressing, the oxygen levels may be replenished.

The inventor provides several different ways in which the oxygen level of a diffusion wound dressing may be determined. In one embodiment a globule of a gelatinous mixture of Methylene Blue is placed between two layers of material (as represented in FIG. 1 and FIG. 3) to form a patch-like indicating device. The oxygen scavenging properties of this compound and the subsequent color change when exposed to oxygen (deep blue color), are well known in the industry. The outer layer of the indicator, being a transparent non-permeable layer, allows a user to clearly identify the color change of the dressing. A gas-permeable layer of the receptacle containing the Methylene Blue is exposed to the inner oxygen rich reservoir of the dressing, providing an intimate contact to oxygen gas trapped in the reservoir. As the oxygen level decreases in the wound dressing reservoir, the Methylene Blue globule experiences de-oxidation. Therefore, the compound will begin to change color to leucomethylene blue, a clear form of the compound. A predetermined color chart may then be compared by a user to approximate the level of oxygen remaining in the reservoir.

In other embodiments of the present invention a single strip 202, with a smaller amount of Methylene Blue, is accommodated in much the same way as described above. In this embodiment a color chart may be incorporated onto the strip, providing the convenience of having both components together (as illustrated in FIG. 2) and then adhered to the underside of the non-permeable outer layer of the dressing.

FIG. 3 is a cross-section representation of wound dressing 101 showing many of the layers and the placement of an oxygen-level indicator 102 described above. An oxygen reservoir 105 is formed between an upper, non-permeable layer of material 103 and a lower oxygen-permeable layer 104. Around the periphery of the dressing the layers 103 and 104 are in intimate contact, but separated in the area desired for the reservoir to create the volume for the reservoir. In some embodiments a “buff-puff” layer 106 is included within the reservoir between the permeable layer and the non-permeable layer. This is a layer of open-celled material shaped in the plan shape of the reservoir, and placed between the permeable and non-permeable layers before these two are pressed and sealed together. The operation of joining the two layers with the open-celled layer between forms the reservoir in this example. In some cases when the reservoir is charged with oxygen or other gas at pressure the reservoir is forced to a larger extent than the original extent of the open-celled layer.

In some embodiments a hydrocolloid layer 107, which is permeable, is added beneath the permeable layer 104 as a protective layer in contact with a patient's skin or a wound. The resulting dressing construct is captured between to layers of release paper to keep the construct clean and protected before use. An adhesive film is added to serve to adhere the dressing to a patient in use. In the embodiment shown in FIG. 3 an oxygen indicator 102 is placed provided to be seen through an opening in the non-permeable upper layer. The container or reservoir for the oxygen indicator has a non-permeable upper portion and a permeable lower portion. The permeable lower portion is in contact with the oxygen in the dressing, and assumes a color corresponding to the percentage of oxygen in the reservoir. In some embodiments the indicator may be a strip as described above.

The dressing may be manufactured with oxygen in the reservoir, or there may be a self-sealing entrance to charge the reservoir as described above. Finally the finished dressing is captured between two layers of release paper to be packaged for transport.

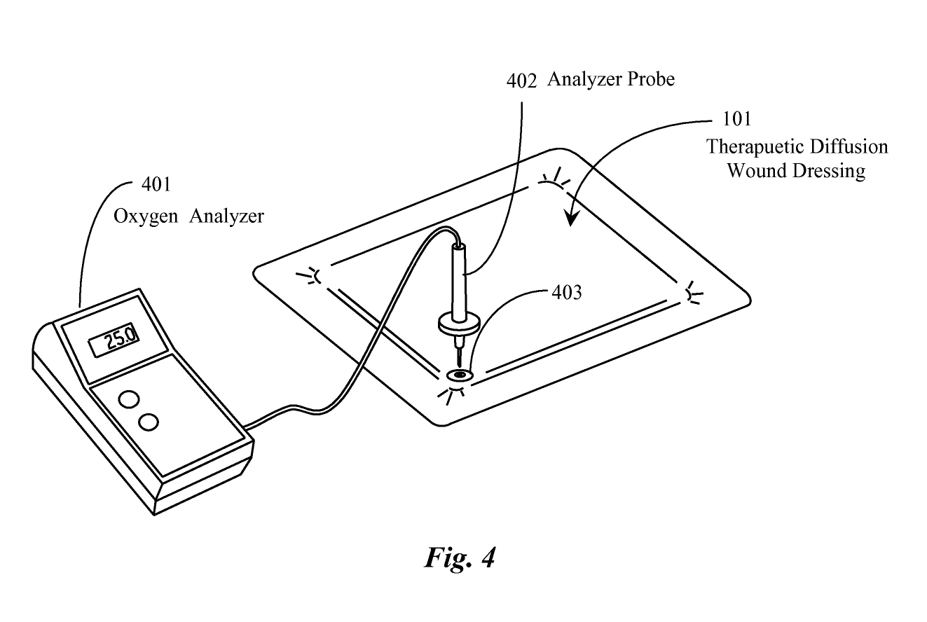

FIG. 4 is an illustration of the therapeutic diffusion wound dressing 101 of FIG. 1 and a separate oxygen analyzing device 401. This device may be utilized to sample a small portion of the oxygen enriched gas, being diffused through the dressing reservoir, to determine the oxygen level. A small diameter probe 402 has a narrow tip to pass through a self-closing soft rubber seal 403 which has been incorporated into the non-permeable layer, in much the same manner as noted above. This seal prevents oxygen-rich gas from escaping from the reservoir during the sensing operation. The gas sampled is then analyzed internally to the device with a readout of the oxygen content of the reservoir displayed to a user. If the reading is below a predetermined level of oxygen, the dressing may be removed and replaced with a fresh one, or replenished with a fresh supply of oxygen gas through the same soft rubber seal 403.

The wound dressings of the present disclosure may also provide a moist environment which facilitates a normal wound-healing process. The inventor indicates common usage for the therapeutic diffusion hydrocolloid wound dressing of FIG. 1 to be used to cover and protect wounds and catheter sites, or they may also be used as a secondary dressing for other wound products such as gauze, alginates, hydrogels, debridement facilitators or a protective cover over at-risk skin. But more specifically, the wound dressing is indicated for such as clean, closed surgical incisions, skin graft donor sites, Stage I or stage II pressure ulcers, pressure sores, superficial wounds such as abrasions, skin tears, blisters, lacerations, first and second degree burns, chafed skin, skin continuously exposed to moisture, secondary dressing over gauze and alginates or hydrogels.

The finished dressing may be placed in a high oxygen barrier pouch, vacuum evacuated and oxygen flushed using a bench-top vacuum/gas sealer using 99.0% USP grade oxygen. Pouch and dressing are allowed sufficient time to equilibrate at a target oxygen concentration exceeding 90%. To allow proper equilibration the lower release paper and hydrocolloid layer will have perforations or holes to allow a gas path from the sealed pouch to the oxygen reservoir in the dressing.

It will be apparent to a skilled artisan that the embodiments described above are exemplary of inventions that may have greater scope than any of the singular descriptions. There may be many alterations made in these examples without departing from the spirit and scope of the invention. For example, the size and shape may vary widely; depending upon the area of the body the dressing may be applied. The material compositions of the oxygen indicating devices and or the wound dressings layer construction and composition, as illustrated in this disclosure, may vary depending on the application area and the environment and therapeutic agents to which they may be exposed. These and many other features may change in different embodiments.

1. A therapeutic wound dressing, comprising:
a reservoir having a two-dimensional footprint formed between an upper layer not permeable to gases, and a lower layer permeable to gases;
an adhesion layer overlapping periphery of the upper, non-permeable layer and extending beyond periphery of the two-dimensional footprint, the adhesion material facing in the direction of the permeable layer, such that the adhesion layer may be pressed to skin of a patient around a wound, sealing the dressing to the patient with the permeable layer directly over the wound; and
one or the other of an oxygen-level indicator containing a material exhibiting color change with varying concentration of oxygen, the indicator placed to be visible to a person viewing the dressing through an interface not permeable to oxygen, and to be open to oxygen through a second interface in intimate contact with oxygen in the reservoir, or a self-sealing opening through which a hollow tube may pass without allowing oxygen to escape, the self-sealing opening useful for charging the reservoir with oxygen at pressure, or for sampling oxygen in the reservoir by an external probe.

2. The wound dressing of claim 1 wherein the oxygen level indicator comprises a gelatinous material Methylene Blue.

3. The wound dressing of claim 1 wherein the oxygen level indicator is a strip of material also comprising a color chart indication oxygen concentration relative to color.

4. The wound dressing of claim 1 packaged between two layers of release paper for storage or transport.

5. A method for monitoring oxygen level in an oxygen reservoir of a therapeutic wound dressing, comprising the steps of:
(a) positioning one or the other of an oxygen level indicator containing a material exhibiting color change with varying concentration of oxygen, the indicator placed to be visible to a person viewing the dressing through an interface not permeable to oxygen, and to be open to oxygen through a second interface in intimate contact with oxygen in the reservoir, or a self-sealing opening into the reservoir through which a hollow tube may pass without allowing oxygen to escape;
(b) if the dressing comprises the visible oxygen-level indicator, viewing the indicator and comparing color of the indicator with a reference to determine oxygen level in the reservoir; and
(c) if the reservoir comprises the self-sealing opening, passing a hollow probe tip through the opening, collecting a sample of atmosphere in the reservoir for analysis for oxygen concentration.

6. The method of claim 5 wherein the oxygen level indicator comprises a gelatinous material Methylene Blue.

7. The method of claim 5 wherein the oxygen level indicator is a strip of material also comprising a color chart indication oxygen concentration relative to color.

8. The method of claim 5 wherein the wound dressing is packaged between two layers of release paper for storage or transport.
